(19) United States
(12) Patent Application Publication
(10) Pub. No.: US 2022/0241159 A1
(43) Pub. Date: Aug. 4, 2022

(54) COSMETIC COMPOSITIONS IN A SOFT-SOLID FORMAT COMPRISING A HYDROPHOBIC POWDER SELECTED FROM SILICA AEROGEL AND POLYLACTIC ACID, STARCH AND AN UV-FILTER SYSTEM

(71) Applicant: L'OREAL, Paris (FR)

(72) Inventors: Bruna RODRIGUES SALOMAO, Rio de Janeiro (BR); Lais MOREIRA LIMA, Rio de Janeiro (BR); Angeles FONOLLA-MORENO, Rio de Janeiro (BR)

(21) Appl. No.: 17/594,739

(22) PCT Filed: Apr. 30, 2019
(86) PCT No.: PCT/BR2019/050160

§ 371 Date: Oct. 28, 2021

(51) Int. Cl. A61K 8/25; A61K 8/02; A61K 8/34; A61K 8/37; A61K 8/39; A61K 8/85; A61K 8/86; A61Q 17/04

(52) U.S. Cl. CPC A61K 8/25; A61K 8/0216; A61K 8/342; A61Q 17/04; A61K 8/39; A61K 8/85; A61K 8/86; A61K 8/375

(57) ABSTRACT

The present invention relates to a new galenic formulation of cosmetic compositions in a soft-solid format, containing high concentration of hydrophobic compounds and high SPF values. The new galenic formulation has a higher viscosity than usual products on the market, but having desired spreadability and sensorial properties due to its unique rheology behavior. The cosmetic composition of the present invention presents enhanced anti-oiliness and anti-acne effects, as well as a dry touch after application, due to its high concentration of hydrophobic compounds.

### FIELD OF THE INVENTION

The present invention relates to a new galenic formulation of cosmetic compositions in a soft-solid format, containing high concentration of hydrophobic compounds and high SPF values. The new galenic formulation has a higher viscosity than usual products on the market, but having desired spreadability and sensorial properties due to its unique rheology behavior. The cosmetic composition of the present invention presents enhanced anti-oiliness and anti-acne effects, as well as a dry touch after application, due to its high concentration of hydrophobic compounds.

### BACKGROUND OF THE INVENTION

The photoprotection of keratinous materials, including both skin and hair, is considered of great importance in order to protect from sun-damage, sunburn, photo-aging, as well as to decrease the chances of skin cancer development caused by exposure to ultraviolet (“UV”) radiation. There are typically two types of UVA/UVB sunscreen compositions used to accomplish photoprotection, namely, inorganic UV filters and organic UV filters.

The degree of UV protection afforded by a sunscreen composition is directly related to the amount and type of UV filters contained therein. The higher the amount of UV filters, the greater the degree of UV protection (UVA/UVB).

Particularly, sunscreen compositions must provide good protection against the sun, a measure of which is the Sun Protection Factor (SPF) value, yet have satisfactory sensory perception, such as a good spreadability and dry touch after application, thereby being smooth but not greasy feel upon application.

The hydrophobic powders such as silica and polylactic acid particles are known in the cosmetic industry for providing anti-oiliness and anti-acne effects in sunscreen compositions. However, the maximum concentration of hydrophobic powders in a sunscreen composition is limited, since the high hydrophobic properties renders the composition instable when high concentrations are applied. In this sense, sunscreen compositions are usually limited, for example, to have about 0.5% by weight of silica, relative to the total weight of the composition.

Thus, there has been a need for sunscreen compositions having higher concentrations of hydrophobic powders, in order to enhance the anti-oiliness and anti-acne effects thereof. The challenge of incorporating high concentrations of hydrophobic powders in the sunscreen composition is not only limited due to their hydrophobic nature, but there is also the challenge on formulating stable compositions while preserving satisfactory properties of the product, such as spreadability, high SPF and dry touch to the skin after application.

Considering the current drawbacks of the state of the art and the difficulties to overcome them, the inventors developed a new galenic formulation of cosmetic compositions in a soft-solid format that enables high concentrations of hydrophobic powders by combining, besides the hydrophobic powder, an UV filter system, at least one water-soluble polysaccharide chosen from starches and at least one nonionic surfactant. The cosmetic composition according to the present invention surprisingly showed enhanced anti-oiliness and anti-acne effects, good spreadability, high SPF values and good sensorial properties, such as dry touch to the skin after application. The pH achieved by the cosmetic composition of the present invention is more acid than the cosmetic compositions of the prior art, thereby allowing different applications, for example, employing acidic active compounds, which can be introduced for providing more benefits to the skin.

Moreover, the soft-solid cosmetic formulation of the present invention presents a unique rheology behavior, wherein the viscosity is higher than the similar products on the market and the viscosity profile is similar to the cosmetic compositions of the prior art, but higher consistencies are obtained and different yield stress points and strain sweep transitions are achieved, which brings unique textures properties to the composition, such as better spreadability.

Thus, the inventors succeeded to overcome the problems of the state of the art and surprisingly revealed a new galenic formulation for cosmetic compositions in a soft-solid format with high concentrations of hydrophobic powders and high SPF values.

### SUMMARY OF THE INVENTION

The present invention is directed to a new cosmetic composition comprising (a) at least one nonionic surfactant, (b) a hydrophobic powder selected from silica aerogel and polylactic acid (PLA), (c) at least one water-soluble polysaccharide, and (d) UV filter system, wherein the cosmetic composition is in a soft-solid format.

The present invention also relates to the use of the cosmetic composition for the manufacture of a product to be used as sunscreen daily product.

### BRIEF DESCRIPTION OF THE DRAWINGS

FIG. 1 shows the rheology analysis results for a composition according to the present invention compared with compositions according to the prior art.

### DETAILED DESCRIPTION OF THE INVENTION

In an embodiment, the cosmetic composition of the present invention comprises:

(a) at least about 1% by weight, based on the total weight of the composition, of at least one nonionic surfactant;

(b) at least about 1% by weight, based on the total weight of the composition of a hydrophobic powder selected from the group consisting of silica aerogel and polylactic acid (PLA);

(c) at least about 1% by weight, based on the total weight of the composition, of at least one water-soluble polysaccharide chosen from starches;

(d) UV filter system,

wherein the composition is in a soft-solid format.

The cosmetic composition of the present invention is presented in a new galenic formulation, in a soft-solid format, having a unique rheology behavior that confer differential texture properties thereto, such as better spreadability and pleasant sensorial to the skin, also achieving high concentration of hydrophobic powders that confer enhanced anti-oiliness and anti-acne effects and dry touch to the skin after application.

In a preferred embodiment, the amount of the at least one nonionic surfactant in the cosmetic composition of the present invention ranges from about 1% to 20% by weight and preferably from about 1% to about 15% by weight, more preferably from about 1% to about 10% by weight, based on the total weight of the composition.

In a preferred embodiment, the at least one nonionic surfactant of the present invention is selected from the group consisting of steareth-2, steareth-20, glyceryl stearate, PEG-100 stearate and cetearyl alcohol.

In a preferred embodiment, the cosmetic composition of the present invention has an amount of UV filter system from about 0.1% to about 50% by weight, preferably in an amount of from about 5% to about 40% by weight, more preferably about 5% to about 30% by weight, most preferably about 10% to about 30% by weight, based on the total weight of the composition.

The UV filter system of the present invention may comprise at least one UV filter selected from the group of inorganic UV filters and organic UV filters, and mixtures thereof. In a preferred embodiment, the UV filters of the present invention are select from the group consisting of butyl methoxydibenzoylmethane, ethylhexyl salicylate, ethylhexyl triazone, octocrylene, homosalate and mixtures thereof.

The amount of the hydrophobic powder in the cosmetic composition of the invention preferably ranges from about 1% to about 10% by weight, more preferably from about 1% to about 8% by weight, and most preferably from about 2% to about 8% by weight, based on the total weight of the composition.

In a preferred embodiment, the amount of the at least one water-soluble polysaccharide in the cosmetic composition of the present invention ranges from about 0.5% to about 10% by weight and preferably from about 1% to about 8% by weight, more preferably from about 1% to about 5% by weight, based on the total weight of the composition.

The at least one water-soluble polysaccharide of the present invention is selected from starches, preferably selected from the group consisting of monostarch phosphates, distarch phosphates and tristarch phosphates. In a more preferred embodiment, the at least one water-soluble polysaccharide is hydroxypropyl starch phosphate.

The cosmetic composition of the invention is in the form of an oil in water (O/W) emulsion and can be used as a daily product for the skin.

The pH of the cosmetic composition according to the present invention is generally between 4 and 6.

In a preferred embodiment, the cosmetic composition of the present invention is a sunscreen composition. In another preferred embodiment, the composition of the present invention presents a SPF of 30, 50, 60, 70, 90 or 100.

Preferably, the soft-solid cosmetic composition according to the present invention has a viscosity of 3500 to 6000 m·Pa, more preferably of 4000 to 5500 m·Pa, measured at 25° C. and 1 atm.

In another preferred embodiment, the present invention is related to the use of a composition for manufacturing a product for preventing sunburn, which can be used as sunscreen daily product.

### Terms

As used herein, the expression “at least” means one or more and thus includes individual components as well as mixtures/combinations.

Other than in the operating examples, or where otherwise indicated, all numbers expressing quantities of ingredients and/or process conditions are to be understood as being modified in all instances by the term “about,” meaning within +/−5% of the indicated number.

As used herein, all ranges provided are meant to include every specific range within, and combination of sub ranges between, the given ranges. Thus, a range from 1-5, includes specifically 1, 2, 3, 4 and 5, as well as sub ranges such as 2-5, 3-5, 2-3, 2-4, 1-4, etc. All ranges and values disclosed herein are inclusive and combinable. For examples, any value or point described herein that falls within a range described herein can serve as a minimum or maximum value to derive a sub-range, etc.

Particularly, for the purposes of the present invention, the term “soft-solid” relates to emulsions that tend to be in the solid state, but presents fluidity when a force is applied. When compared to conventional liquid emulsions, a soft-solid emulsion has the same viscosity behavior, but presents higher consistency.

### Surfactants

Non-Ionic Surfactants

The cosmetic composition according to the present invention comprises at least one nonionic surfactant,

Non-limiting examples of non-ionic surfactants useful in the present invention include, for example, alkyl- and polyalkyl-esters of glycerol, such as polyglyceryl-3 dicitrate/stearate, mixtures of alkyl- and polyalkyl-esters of glycerol with polyglyceryl, such as polyglyceryl-3 methylglucose distearate, oxyalkylenated (more particularly polyoxyethylenated) fatty acid esters of glycerol; oxyalkylenated fatty acid esters of sorbitan; oxyalkylenated (oxyethylenated and/or oxypropylenated) fatty acid esters; oxyalkylenated (oxyethylenated and/or oxypropylenated) fatty alcohol ethers; sugar esters, for instance sucrose stearate; fatty alcohols, fatty alcohol ethers of sugars, especially alkyl polyglucosides (APGs) such as decyl glucoside, lauryl glucoside, cetostearyl glucoside, optionally as a mixture with cetostearyl alcohol, and also arachidyl glucoside, for example in the form of a mixture of arachidyl alcohol, behenyl alcohol and arachidyl glucoside. According to one particular embodiment of the invention, the mixture of the alkyl polyglucoside as defined above with the corresponding fatty alcohol may be in the form of a self-emulsifying composition. Mention may also be made of lecithins and derivatives (e.g. Biophilic), sugar esters and sodium stearoyl lactylate.

In a preferred embodiment, the at least one nonionic surfactant of the present invention is select from the group consisting of steareth-2, steareth-20, glyceryl stearate, PEG-100 stearate and cetearyl alcohol.

Preferably, the amount of the at least one nonionic surfactant in the cosmetic composition of the present invention ranges from about 1% to 20% by weight and preferably from about 1% to about 15% by weight, more preferably from about 1% to about 10% by weight, based on the total weight of the composition.

Additional Surfactants

The cosmetic composition of the present invention may also comprise at least one additional surfactant, preferably, at least one anionic surfactants.

Anionic surfactants mean a surfactant comprising, as ionic or ionizable groups, only anionic groups. These anionic groups are chosen preferably from the groups CO₂H, CO₂—, SO₃H, SO₃—, OSO₃H, OSO₃—O₂PO₂H, O₂PO₂H and O₂PO₂²⁻.

Non-limiting anionic surfactant(s) that may be used in the present invention are selected from the group comprising alkyl sulfates, alkyl ether sulfates, alkylamido ether sulfates, alkylaryl polyether sulfates, monoglyceride sulfates, sulfonates, such as alkylsulfonates, alkylamide sulfonates, alkylarylsulfonates, alpha-olefin sulfonates, paraffin sulfonates, sulfosuccinates, alkylsulfosuccinates, alkyl ether sulfosuccinates, alkylamide sulfosuccinates, alkyl sulfoacetates, acylsarcosinates, acylglutamates, alkylsulfosuccinamates, taurates and N-acyl N-methyltaurates, isethionates, N-acylisethionates, N-acyltaurates, phosphates and alkyl phosphates, salts of alkyl monoesters and polyglycoside-polycarboxylic acids, acyllactylates, mixed esters of organic acids with glycerol, such as glyceryl stearate citrate and as glyceryl stearate lactate, salts of D-galactoside uronic acids, salts of alkyl ether carboxylic acids, salts of alkyl aryl ether carboxylic acids, and salts of alkylamido ether carboxylic acids; or the non-salified forms of all of these compounds, the alkyl and acyl groups of all of these compounds containing from 6 to 24 carbon atoms and the aryl group denoting a phenyl group. Some of these compounds may be oxyethylenated and then preferably comprise from 1 to 50 ethylene oxide units.

### Hydrophobic Powders

The cosmetic composition according to the present invention comprises a hydrophobic powder selected from the group consisting of silica aerogel and polylactic acid (PLA).

The amount of the hydrophobic powder in the cosmetic composition of the invention is at least about 1% by weight, based on the total weight of the composition, preferably ranging from about 1% to about 10% by weight, more preferably from about 1% to about 8% by weight, and most preferably from about 2% to about 8% by weight, based on the total weight of the composition.

Silica Aerogel

The “silica aerogel” according to the present invention is a porous material obtained by replacing (by drying) the liquid component of a silica gel with air. Silica aerogels are generally synthesized via a sol-gel process in a liquid medium and then dried, usually by extraction with a supercritical fluid, such as, but not limited to, supercritical carbon dioxide (CO₂). This type of drying makes it possible to avoid shrinkage of the pores and of the material. The sol-gel process and the various drying processes are described in detail in Brinker, C. J., and Scherer, G. W., Sol-Gel Science: New York: Academic Press, 1990.

The hydrophobic silica aerogel particles used in the present invention have a specific surface area per unit of mass (SM) ranging from about 500 to about 1500 m²/g, or alternatively from about 600 to about 1200 m²/g, or alternatively from about 600 to about 800 m²/g, and a size expressed as the mean volume diameter (D[0.5]), ranging from about 1 to about 30 μm, or alternatively from about 5 to about 25 μm, or alternatively from about 5 to about 20 μm, or alternatively from about 5 to about 15 μm. The specific surface area per unit of mass may be determined via the BET (Brunauer-Emmett-Teller) nitrogen absorption method described in the Journal of the American Chemical Society, vol. 60, page 309, February 1938, corresponding to the international standard ISO 5794/1. The BET specific surface area corresponds to the total specific surface area of the particles under consideration.

The size of the silica aerogel particles may be measured by static light scattering using a commercial granulometer such as the MasterSizer 2000 machine from Malvern. The data are processed on the basis of the Mie scattering theory. This theory, which is exact for isotropic particles, makes it possible to determine, in the case of non-spherical particles, an “effective” particle diameter. This theory is especially described in the publication by Van de Hulst, H. C., “Light Scattering by Small Particles,” Chapters 9 and 10, Wiley, New York, 1957.

The silica aerogel particles used in the present invention may advantageously have a tamped (or tapped) density ranging from about 0.04 g/cm³ to about 0.10 g/cm³, or alternatively from about 0.05 g/cm³ to about 0.08 g/cm³. In the context of the present invention, this density, known as the tamped density, may be assessed according to the following protocol: 40 g of powder are poured into a measuring cylinder; the measuring cylinder is then placed on a Stay 2003 machine from Stampf Volumeter; the measuring cylinder is then subjected to a series of 2500 packing motions (this operation is repeated until the difference in volume between two consecutive tests is less than 2%); the final volume Vf of packed powder is then measured directly on the measuring cylinder. The tamped density is determined by the ratio m/Vf, in this instance 40/Vf (Vf being expressed in cm³ and m in g).

According to one embodiment, the hydrophobic silica aerogel particles used in the present invention have a specific surface area per unit of volume Sv ranging from about 5 to about 60 m²/cm³, or alternatively from about 10 to about 50 m²/cm³, or alternatively from about 15 to about 40 m²/cm³. The specific surface area per unit of volume is given by the relationship: Sv=S_{M}·r where r is the tamped density expressed in g/cm³ and S_{M} is the specific surface area per unit of mass expressed in m²/g, as defined above.

In some embodiments, the silica aerogel particles, according to the invention, have an oil-absorbing capacity, measured at the wet point, ranging from about 5 to about 18 ml/g, or alternatively from about 6 to about 15 ml/g, or alternatively from about 8 to about 12 ml/g. The oil-absorbing capacity measured at the wet point, noted Wp, corresponds to the amount of water that needs to be added to 100 g of particle in order to obtain a homogeneous paste. Wp is measured according to the wet point method or the method for determining the oil uptake of a powder described in standard NF T 30-022. Wp corresponds to the amount of oil adsorbed onto the available surface of the powder and/or absorbed by the powder by measuring the wet point, described below: An amount=2 g of powder is placed on a glass plate, and the oil (isononyl isononanoate) is then added dropwise. After addition of 4 to 5 drops of oil to the powder, mixing is performed using a spatula, and addition of oil is continued until a conglomerate of oil and powder has formed. At this point, the oil is added one drop at a time and the mixture is then triturated with the spatula. The addition of oil is stopped when a firm, smooth paste is obtained. This paste must be able to be spread on the glass plate without cracking or forming lumps. The volume Vs (expressed in ml) of oil used is then noted. The oil uptake corresponds to the ratio Vs/m.

The aerogels used, according to the present invention, are hydrophobic silica aerogels, preferably of silylated silica (INCI name: silica silylate). The term “hydrophobic silica” means any silica whose surface is treated with silylating agents, for example, halogenated silanes, such as alkylchlorosilanes, siloxanes, in particular dimethylsiloxanes, such as hexamethyldisiloxane, or silazanes, so as to functionalize the OH groups with silyl groups Si—Rn, for example, trimethylsilyl groups. Preparation of hydrophobic silica aerogel particles that have been surface-modified by silylation, is found in U.S. Pat. No. 7,470,725, incorporated herein by reference. In one embodiment, hydrophobic silica aerogel particles surface-modified with trimethylsilyl groups are desirable.

Suitable examples of hydrophobic silica aerogels, may include, but are not limited to, the aerogels sold under the tradenames of VM-2260 (INCI name: Silica silylate) and VM-2270 (INCI name: Silica silylate), both available from Dow Corning Corporation (Midland, Mich.). The particles of VM-2260 have a mean size of about 1000 microns and a specific surface area per unit of mass ranging from 600 to 800 m²/g. The particles of VM-2270 have a mean size ranging from 5 to 15 microns and a specific surface area per unit of mass ranging from 600 to 800 m²/g. Another suitable example of a hydrophobic silica aerogel may include, but is not limited to, the aerogels commercially available from Cabot Corporation (Billerica, Mass.) under the tradename of Aerogel TLD 201, Aerogel OGD 201 and Aerogel TLD 203, Enova Aerogel MT 1100 and Enova Aerogel MT 1200.

The silica aerogel is preferably hydrophobic silica aerogel, more preferably silica silylate.

Polylactic Acid (PLA)

The cosmetic composition of the invention may comprise porous microparticles of a polylactic acid-based resin, sometimes referred to herein as “polylactic acid”, “polylactic acid microparticles” or “PLA.”

The PLA microparticles may have an enthalpy of fusion of 5 J/g or more, preferably 10 J/g or more, more preferably 20 J/g or more, and most preferably 30 J/g or more. Further, the upper limit is preferably 100 J/g or less, although it is not limited in particular. Enthalpy of fusion refers to a value calculated from a peak area, which shows heat capacity of fusion at approximately 160° C., in a differential scanning calorimetry (DSC) where a temperature is raised to 200° C. with the temperature rise of 20° C. per minute.

Enthalpy of fusion can be adjusted by controlling the co-polymerization ratio (L/D) between L-lactic acid and D-lactic acid which constitute the polylactic acid-based resin. When the L/D ratio is 95/5 or more, enthalpy of fusion becomes 5 J/g or more and the polylactic acid-based resin becomes crystalline. It is preferred that the co-polymerization ratio of L-lactic acid is high because higher ratios facilitate crystallization. L/D is more preferably 97/3 or more, and most preferably 98/2 or more. L/D is 100/0 or less. Because optical isomers such as L and D have molecular structures that are mirror images of each other and physical properties are not different, enthalpy of fusion remains unchanged when the above-described L/D is substituted with D/L and consequently suitable resins include ones in which L/D is substituted with D/L.

Further, the polylactic acid-based resin may contain copolymerization ingredients other than lactic acid. The other copolymerization ingredient units can be, for example, a multivalent carboxylic acid, a polyhydric alcohol, a hydroxycarboxylic acid or a lactone. Exemplary multivalent carboxylic acids include oxalic acid, malonic acid, succinic acid, glutaric acid, adipic acid, azelaic acid, sebacic acid, dodecanedioic acid, fumaric acid, cyclohexanedicarboxylic acid, terephthalic acid, isophthalic acid, phthalic acid, 2,6-naphthalenedicarboxylic acid, anthracene dicarboxylic acid, 5-sodium sulfoisophthalic acid and 5-tetrabutyl phosphonium sulfoisophthalic acid. Exemplary polyhydric alcohols include ethylene glycol, propylene glycol, butanediol, heptanediol, hexanediol, octanediol, nonanediol, decanediol, 1,4-cyclohexanedimethanol, neopentyl glycol, glycerin, pentaerythritol, bisphenol A, an aromatic polyhydric alcohol produced by an addition reaction of ethylene oxide to a bisphenol, diethylene glycol, triethylene glycol, polyethylene glycol, polypropylene glycol and polytetramethylene glycol. Exemplary hydroxycarboxylic acids include glycolic acid, 3-hydroxybutyric acid, 4-hydroxybutyric acid, 4-hydroxyvaleric acid, 6-hydroxycaproic acid and hydroxybenzoic acid. Exemplary lactones include glycolide, ε-caprolactone glycolide, ε-caprolactone, β-propiolactone, δ-butyrolactone, β-butyrolactone, γ-butyrolactone, pivalolactone and δ-valerolactone. The volume content of the other copolymerization units is preferably 30 mol % or less, more preferably 20 mol % or less, further more preferably 10 mol % or less, most preferably 5 mol % or less, relative to the total monomer units of the polylactic acid-based resin as 100 mol %.

Although molecular mass and molecular mass distribution of the polylactic acid-based resin are not limited in particular, the lower limit of weight average molecular mass of the polylactic acid-based resin is preferably 10,000 or more, more preferably 50,000 or more, further more preferably 100,000 or more, most preferably 200,000 or more. Further, although not limited in particular, the upper limit of weight average molecular mass is preferably 1,000,000 or less. The weight average molecular mass referred to herein is weight average molecular mass in terms of polymethyl methacrylate (PMMA), measured by gel permeation chromatography (GPC) using hexafluoroisopropanol as a solvent.

The PLA microparticles may have a number average particle diameter of 90 μm or less, preferably 50 μm or less, more preferably 30 μm or less. This improves smoothness. Further, in uses such as cosmetics, because coagulation of particles tends to occur when the number average particle diameter is too small, the lower limit of the number average particle diameter is generally 1 μm or more, preferably more than 1 μm, more preferably 2 μm or more, most preferably 3 μm or more.

The particle diameter distribution index is preferably 2 or less in order to improve flow of the particles and impart a smoother touch. The upper limit of the particle diameter distribution index is preferably 1.5 or less, more preferably 1.3 or less, most preferably 1.2 or less. Further, the lower limit is 1 in theory.

The above-described number average particle diameter of polylactic acid-based resin microparticles having porous shapes can be calculated by measuring diameters of 100 random particles in a scanning electron microscope image and computing the arithmetic average thereof. If a shape of a particle in the SEM image is not a perfect circle, for example, an ellipse, the maximum diameter of the particle is used as its diameter. To measure the particle diameter precisely, the measurement is carried out with a magnification of at least 1000 times or more, preferably with a magnification of 5000 times or more.

The particle diameter distribution index is calculated on the basis of the conversion equations described below, using measurements of the particle diameters obtained by measurement described above:

$$Dn = {\sum }_{i = 1}^{n} Ri / n$$

$$Dv = {\sum }_{i = 1}^{n} {Ri}^{4} / {\sum }_{i = 1}^{n} {Ri}^{3}$$

$$PDI = Dv / Dn$$

wherein Ri: particle diameter of single particle, n: the number of measurements (=100), Dn: number average particle diameter, Dv: volume average particle diameter, PDI: particle diameter distribution index.

Although the actual amount of pores in a porous microparticle of polylactic acid-based resin is difficult to measure directly, it is possible to use linseed oil absorption capacity as an indirect index, which is defined in pigment test methods such as Japan Industrial Standards (Refined Linseed Oil Method, JIS K 5101).

In particular, in the uses such as cosmetics and paints, higher linseed oil capability is preferable, and the lower limit of linseed oil capability is preferably 90 mL/100 g or more, more preferably 100 mL/100 g or more, further more preferably 120 mL/100 g or more, particularly preferably 150 mL/100 g or more, remarkably preferably 200 mL/100 g or more, most preferably 300 mL/100 g or more. The upper limit of linseed oil absorption capability is preferably 1000 mL/100 g or less.

Further, it is preferred that the above-described porous microparticles of polylactic acid-based resin have enthalpy of fusion of 5 J/g or more. Higher enthalpy of fusion brings higher crystallization tendency and, as a result, heat resistance and durability tend to become high. The lower limit of enthalpy of fusion is preferably 10 J/g or more, more preferably 20 J/g or more, further more preferably 30 J/g or more. Further, the upper limit is preferably 100 J/g or less. Enthalpy of fusion can be calculated from an area of peak showing thermal capacity of fusion at approximately 160° C. in Differential Scanning calorimetry (DSC) in which a temperature is raised to 200° C. with a temperature rise of 20° C. per minute.

Sphericity of the above-described porous microparticles of polylactic acid-based resin is preferably 80 or more, more preferably 85 or more, furthermore preferably 90 or more, particularly preferably 92 or more, most preferably 95 or more. Further, in theory, the upper limit is 100. When sphericity is within the above-described range, it becomes possible to achieve an improvement in quality such as slidability. The sphericity is calculated by observing particles by a scanning electron microscope, measuring both the longest diameters and the shortest diameters of 30 random particles and subsequently substituting the measurements into the equation described below:

$$S = \frac{{\sum }_{i = 1}^{n} ( {D}_{S} / {D}_{L} )}{n} \times 100$$

wherein S: Sphericity, n: the number of measurements (=30), Ds: the shortest diameter of single particle, DL: the longest diameter of single particle.

### Water-Soluble Polysaccharides

The cosmetic composition according to the present invention comprises at least one water-soluble polysaccharide chosen from starches. The term “polysaccharide” means any polymer consisting of several saccharides (or monosaccharides) having the general formula:

—[Cₓ(H2O)_{y})]ₙ— (in which y is generally x−1)

and linked together via O-oside bonds.

The at least one water-soluble polysaccharide to be used in the present invention is chosen from starches, and mixtures thereof.

The term “water-soluble” means partially or totally soluble in water to give a gelled or thickened solution at a concentration of 1% active material in water, after implementation with or without heating.

The starches to be used in the present invention are more particularly macromolecules in the form of polymers formed from elemental units that are anhydroglucose units. The number of these units and their assembly make it possible to distinguish amylose (linear polymer) and amylopectin (branched polymer). The relative proportions of amylose and of amylopectin, and their degree of polymerization, vary as a function of the botanical origin of the starches. The amylose/amylopectin weight ratio may range from 30/70 (corn) to 16/84 (rice). The molecular weight of the amylose is preferably up to 1 million by weight and that of the amylopectin is preferably from 100 to 500 million by weight.

The starch molecules used in the present invention may be unmodified or chemically or physically modified.

Their botanical origin may be cereals or tubers. Thus, the natural starches may be chosen from corn starch, rice starch, tapioca starch, cassava starch, barley starch, potato starch, wheat starch, sorghum starch, palm starch and pea starch.

Among the unmodified starches, mention may be made of unmodified corn starches (INCI name: Zea mays starch), for example, products sold under the trade name Farmal CS®, in particular the commercial product Farmal CS 3650® from the company Corn Products International.

Mention may also be made of unmodified rice starches (INCI name: Oryza sativa (rice) starch), for example, the commercial product Remy DR I® sold by the company Beneo-Remy.

According to a particular form of the invention, starches used are modified by crosslinking with functional agents capable of reacting with the hydroxyl groups of the starch molecules, which will thus bond together (for example, with glyceryl and/or phosphate groups).

Monostarch phosphates (of the type St-O—PO—(OX)₂), distarch phosphates (of the type St-O—PO—(OX)—O-St) or even tristarch phosphates (of the type St-O—PO—(O-St)₂) or mixtures thereof may especially be obtained by crosslinking with phosphorus compounds.

X especially denotes alkali metals (for example sodium or potassium), alkaline-earth metals (for example calcium or magnesium), ammonium salts, amine salts, for instance those of monoethanolamine, diethanolamine, triethanolamine, 3-amino-1,2-propanediol, or ammonium salts derived from basic amino acids such as lysine, arginine, sarcosine, ornithine or citrulline.

The phosphorus compounds may be, for example, sodium tripolyphosphate, sodium orthophosphate, phosphorus oxychloride or sodium trimetaphosphate.

Use will preferentially be made of distarch phosphates or of compounds rich in distarch phosphate, in particular the distarch phosphate hydroxypropyl ethers having the INCI name: Hydroxypropyl Starch Phosphate, for example, the products sold under the trade names Farinex VA70 C or Farmal MS 689® from the company Avebe Stadex; the products sold under the trade names Structure BTC®, Structure HVS®, Structure XL® or Structure Zea® from National Starch (corn distarch phosphate).

Preferentially, the starch will be chosen from unmodified corn starches, unmodified rice starches and distarch phosphates, or mixtures thereof.

Even more preferentially, starch will be chosen from distarch phosphates.

In a preferred embodiment, the amount of the at least one water-soluble polysaccharide in the cosmetic composition of the present invention is at least about 0.5% by weight, based on the total weight of the composition, preferably ranging from about 0.5% to 10% by weight and preferably from about 1% to about 8% by weight, more preferably from about 1% to about 5% by weight, based on the total weight of the composition.

### UV Filter System

The composition, according to the present invention, comprises a UV filter system. The UV filter system may comprise at least one UV filter selected from the group of inorganic UV filters and organic UV filters, and mixtures thereof.

The composition according to the present invention, may comprise the UV filter system in an amount of from about 0.1% by weight to about 50% by weight, and in some embodiments from about 5% by weight to about 40% by weight, and in some embodiments from about 5% by weight to about 30% by weight in relation to the total weight of the composition.

Inorganic UV Filters

The composition, according to the present invention, comprise a UV filter system comprising at least one inorganic UV filter. If two or more inorganic UV filters are used, they may be the same or different.

The inorganic UV filter used for the present invention may be active in the UV-A and/or UV-B region. The inorganic UV filter may be hydrophilic and/or lipophilic. The inorganic UV filter is in some embodiments insoluble in solvents, such as water, and ethanol commonly used in cosmetics.

It is in some embodiments desirable that the inorganic UV filter be in the form of a fine particle such that the mean (primary) particle diameter thereof ranges from about 1 nm to about 50 nm, and in some embodiments from about 5 nm to about 40 nm, and in some embodiments from about 10 nm to about 30 nm. The mean (primary) particle size or mean (primary) particle diameter here is an arithmetic mean diameter.

The inorganic UV filter can be selected from the group consisting of silicon carbide, metal oxides which may or may not be coated, and mixtures thereof. And in some embodiments, the inorganic UV filters are selected from pigments (mean size of the primary particles: generally from about 5 nm to about 50 nm, and in some embodiments from about 10 nm to about 50 nm) formed of metal oxides, such as, for example, pigments formed of titanium oxide (amorphous or crystalline in the rutile and/or anatase form), iron oxide, zinc oxide, zirconium oxide, or cerium oxide, which are all UV photoprotective agents that are well known per se. And in some embodiments, the inorganic UV filters are selected from titanium oxide, zinc oxide, and, in some embodiments, titanium oxide.

The inorganic UV filter may or may not be coated. The inorganic UV filter may have at least one coating. The coating may comprise at least one compound selected from the group consisting of alumina, silica, aluminum hydroxide, silicones, silanes, fatty acids or salts thereof (such as sodium, potassium, zinc, iron, or aluminum salts), fatty alcohols, lecithin, amino acids, polysaccharides, proteins, alkanolamines, waxes, such as beeswax, (meth)acrylic polymers, organic UV filters, and (per)fluoro compounds. It is in some embodiments desirable for the coating to include at least one organic UV filter. As the organic UV filter in the coating, a dibenzoylmethane derivative, such as butyl methoxydibenzoylmethane (Avobenzone) and 2,2′-Methylenebis[6-(2H-Benzotriazol-2-yl)-4-(1,1,3,3-Tetramethyl-Butyl) Phenol] (Methylene Bis-Benzotriazolyl Tetramethylbutylphenol), such as marketed as “TINOSORB M” by BASF, may be desirable.

In a known manner, the silicones in the coating(s) may be organosilicon polymers or oligomers comprising a linear or cyclic and branched or cross-linked structure, of variable molecular weight, obtained by polymerization and/or polycondensation of suitable functional silanes and essentially composed of repeated main units in which the silicon atoms are connected to one another via oxygen atoms (siloxane bond), optionally substituted hydrocarbon radicals being connected directly to said silicon atoms via a carbon atom.

The term “silicones” also encompasses silanes necessary for their preparation, in particular alkylsilanes.

The silicones used for the coating(s) can be and in some embodiments are selected from the group consisting of alkylsilanes, polydialkylsiloxanes, and polyalkylhydrosiloxanes. And in some embodiments still, the silicones are selected from the group consisting of octyltrimethylsilane, polydimethylsiloxanes, and polymethylhydrosiloxanes.

Of course, the inorganic UV filters made of metal oxides may, before their treatment with silicones, have been treated with other surfacing agents, in particular with cerium oxide, alumina, silica, aluminum compounds, silicon compounds, or their mixtures. The coated inorganic UV filter may have been prepared by subjecting the inorganic UV filter to one or more surface treatments of a chemical, electronic, mechano-chemical, and/or mechanical nature with any of the compounds as described above, as well as polyethylenes waxes, metal alkoxides (titanium or aluminum alkoxides), metal oxides, sodium hexametaphosphate, and those shown, for example, in Cosmetics & Toiletries, February 1990, Vol. 105, pp. 53-64.

The coated inorganic UV filters may be titanium oxides coated: with silica, such as the product “Sun veil” from Ikeda, and “Sunsil TIN 50” from Sunjin Chemical; with silica and with iron oxide, such as the product “Sunveil F” from Ikeda; with silica and with alumina, such as the products “Microtitanium Dioxide MT 500 SA” from Tayca, “Tioveil” from Tioxide, and “Mirasun TiW 60” from Rhodia; with alumina, such as the products “Tipaque TTO-55 (B)” and “Tipaque TTO-55 (A)” from Ishihara, and “UVT 14/4” from Kemira; with alumina and with aluminum stearate, such as the product “Microtitanium Dioxide MT 100 T, MT 100 TX, MT 100 Z or MT-01” from Tayca, the products “Solaveil CT-10 W” and “Solaveil CT 100” from Uniqema, and the product “Eusolex T-AVO” from Merck; with alumina and with aluminum laurate, such as the product “Microtitanium Dioxide MT 100 S” from Tayca; with iron oxide and with iron stearate, such as the product “Microtitanium Dioxide MT 100 F” from Tayca; with zinc oxide and with zinc stearate, such as the product “BR351” from Tayca; with silica and with alumina and treated with a silicone, such as the products “Microtitanium Dioxide MT 600 SAS”, “Microtitanium Dioxide MT 500 SAS”, and “Microtitanium Dioxide MT 100 SAS” from Tayca; with silica, with alumina and with aluminum stearate and treated with a silicone, such as the product “STT-30-DS” from Titan Kogyo; with silica and treated with a silicone, such as the product “UV-Titan X 195” from Kemira; with alumina and treated with a silicone, such as the products “Tipaque TTO-55 (S)” from Ishihara or “UV Titan M 262” from Kemira; with triethanolamine, such as the product “STT-65-S” from Titan Kogyo; with stearic acid, such as the product “Tipaque TTO-55 (C)” from Ishihara; or with sodium hexametaphosphate, such as the product “Microtitanium Dioxide MT 150 W” from Tayca. Other titanium oxide pigments treated with a silicone are, and in some embodiments TiO₂ treated with octyltrimethylsilane and for which the mean size of the individual particles is from 25 and 40 nm, such as that marketed under the trademark “T 805” by Degussa Silices, TiO₂ treated with a polydimethylsiloxane and for which the mean size of the individual particles is 21 nm, such as that marketed under the trademark “70250 Cardre UF TiO₂Si₃” by Cardre, and anatase/rutile TiO₂ treated with a polydimethylhydrosiloxane and for which the mean size of the individual particles is 25 nm, such as that marketed under the trademark “Microtitanium Dioxide USP Grade Hydrophobic” by Color Techniques.

And in some embodiments, the following coated TiO₂ can be used as the coated inorganic UV filter: Stearic acid (and) Aluminum Hydroxide (and) TiO₂, such as the product “MT-100 TV” from Tayca, with a mean primary particle diameter of 15 nm; Dimethicone (and) Stearic Acid (and) Aluminum Hydroxide (and) TiO₂, such as the product “S A-TTO-S4” from Miyoshi Kasei, with a mean primary particle diameter of 15 nm; Silica (and) TiO₂, such as the product “MT-100 WP” from Tayca, with a mean primary particle diameter of 15 nm; Dimethicone (and) Silica (and) Aluminum Hydroxide (and) TiO₂, such as the product “MT-Y02” and “MT-Y-110 M3S” from Tayca, with a mean primary particle diameter of 10 nm; Dimethicone (and) Aluminum Hydroxide (and) TiO₂, such as the product “SA-TTO-S3” from Miyoshi Kasei, with a mean primary particle diameter of 15 nm; Dimethicone (and) Alumina (and) TiO₂, such as the product “UV TITAN MI 70” from Sachtleben, with a mean primary particle diameter of 15 nm; and Silica (and) Aluminum Hydroxide (and) Alginic Acid (and) TiO₂, such as the product “MT-100 AQ” from Tayca, with a mean primary particle diameter of 15 nm. In terms of UV filtering ability, TiO₂ coated with at least one organic UV filter is more desirable. For example, Avobenzone (and) Stearic Acid (and) Aluminum Hydroxide (and) TiO₂, such as the product “HXMT-100ZA” from Tayca, with a mean primary particle diameter of 15 nm, can be used.

The uncoated titanium oxide pigments are, for example, marketed by Tayca under the trademarks “Microtitanium Dioxide MT500B” or “Microtitanium Dioxide MT600B”, by Degussa under the trademark “P 25”, by Wacker under the trademark “Oxyde de titane transparent PW”, by Miyoshi Kasei under the trademark “UFTR”, by Tomen under the trademark “ITS” and by Tioxide under the trademark “Tioveil AQ”. The uncoated zinc oxide pigments are, for example, those marketed under the trademark “Z-cote” by Sunsmart; those marketed under the trademark “Nanox” by Elementis; and those marketed under the trademark “Nanogard WCD 2025” by Nanophase Technologies. The coated zinc oxide pigments are, for example, those marketed under the trademark “Oxide Zinc CS-5” by Toshiba (ZnO coated with polymethylhydrosiloxane); those marketed under the trademark “Nanogard Zinc Oxide FN” by Nanophase Technologies (as a 40% dispersion in Finsolv TN, C₁₂-C₁₅ alkyl benzoate); those marketed under the trademark “Daitopersion Zn-30” and “Daitopersion Zn-50” by Daito (dispersions in oxyethylenated polydimethylsiloxane/cyclopolymethylsiloxane comprising 30% or 50% of zinc nano-oxides coated with silica and polymethylhydrosiloxane); those marketed under the trademark “NFD Ultrafine ZnO” by Daikin (ZnO coated with phosphate of perfiuoroalkyl and a copolymer based on perfluoroalkylethyl as a dispersion in cyclopentasiloxane); those marketed under the trademark “SPD-Z1” by Shin-Etsu (ZnO coated with a silicone-grafted acrylic polymer dispersed in cyclodimethylsiloxane); those marketed under the trademark “Escalol Z100” by ISP (alumina-treated ZnO dispersed in an ethylhexyl methoxycinnamate/PVP-hexadecene copolymer/methicone mixture); those marketed under the trademark “Fuji ZnO-SMS-10” by Fuji Pigment (ZnO coated with silica and polymethylsilsesquioxane); and those marketed under the trademark “Nanox Gel TN” by Elementis (ZnO dispersed at 55% in C₁₂-C₁₅ alkyl benzoate with hydroxystearic acid polycondensate). The uncoated cerium oxide pigments are marketed, for example, under the trademark “Colloidal Cerium Oxide” by Rhone-Poulenc.

The uncoated iron oxide pigments are, for example, marketed by Arnaud under the trademarks “Nanogard WCD 2002 (FE 45B)”, “Nanogard Iron FE 45 BL AQ”, “Nanogard FE 45R AQ”, and “Nanogard WCD 2006 (FE 45R)”, or by Mitsubishi under the trademark “TY-220”.

The coated iron oxide pigments are, for example, marketed by Arnaud under the trademarks “Nanogard WCD 2008 (FE 45B FN)”, “Nanogard WCD 2009 (FE 45B 556)”, “Nanogard FE 45 BL 345”, and “Nanogard FE 45 BL”, or by BASF under the trademark “Oxyde de fer transparent”.

Mention may also be made of mixtures of metal oxides, in particular, of titanium dioxide and of cerium dioxide, including a mixture of equal weights of titanium dioxide coated with silica and of cerium dioxide coated with silica, such as marketed by Ikeda under the trademark “Sunveil A”, and also a mixture of titanium dioxide and of zinc dioxide coated with alumina, with silica and with silicone, such as the product “M 261” marketed by Kemira, or coated with alumina, with silica and with glycerol, such as the product “M 211” marketed by Kemira.

Coated inorganic UV filters are desirable, because the UV filtering effects of the inorganic UV filters can be enhanced. In addition, the coating(s) may help uniformly or homogeneously disperse the UV filters in the composition, according to the present invention.

Organic UV Filters

The composition, according to the present invention, comprises a UV filter system comprising at least one organic UV filter. If two or more organic UV filters are used, they may be the same or different.

The organic UV filter used for the present invention may be active in the UV-A and/or UV-B region. The organic UV filter may be hydrophilic and/or lipophilic.

The organic UV filter may be solid or liquid. The terms “solid” and “liquid” mean solid and liquid, respectively, at 25° C. under 1 atm.

The organic UV filter can be selected from the group consisting of anthranilic compounds; dibenzoylmethane compounds; cinnamic compounds; salicylic compounds; camphor compounds; benzophenone compounds; β,β-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

Mention may be made, as examples of the organic UV filter(s), of those denoted below under their INCI names, and mixtures thereof. Anthranilic compounds: menthyl anthranilates, such as marketed under the trademark “Neo Heliopan MA” by Haarmann and Reimer. The dibenzoylmethane compounds: Butyl methoxydibenzoylmethane, such as marketed in particular under the trademark “Parsol 1789” by Hoffmann-La Roche; and isopropyl dibenzoylmethane. Cinnamic compounds: Ethylhexyl methoxycinnamate, such as marketed in particular under the trademark “Parsol MCX” by Hoffmann-La Roche; isopropyl methoxycinnamate; isopropoxy methoxycinnamate; isoamyl methoxycinnamate, such as marketed under the trademark “Neo Heliopan E 1000” by Haarmann and Reimer; cinoxate (2-ethoxyethyl-4-methoxy cinnamate); DEA methoxycinnamate; diisopropyl methylcinnamate; and glyceryl ethylhexanoate dimethoxycinnamate. Salicylic compounds: Homosalate (homomentyl salicylate), such as marketed under the trademark “Eusolex HMS” by Rona/EM Industries; ethylhexyl salicylate, such as marketed under the trademark “Neo Heliopan OS” by Haarmann and Reimer; glycol salicylate; butyloctyl salicylate; phenyl salicylate; dipropyleneglycol salicylate, such as marketed under the trademark “Dipsal” by Scher; and TEA salicylate, such as marketed under the trademark “Neo Heliopan TS” by Haarmann and Reimer. Camphor compounds, in particular, benzylidenecamphor derivatives: 3-benzylidene camphor, such as manufactured under the trademark “Mexoryl SD” by Chimex; 4-methylbenzylidene camphor, such as marketed under the trademark “Eusolex 6300” by Merck; benzylidene camphor sulfonic acid, such as manufactured under the trademark “Mexoryl SL” by Chimex; camphor benzalkonium methosulfate, such as manufactured under the trademark “Mexoryl SO” by Chimex; terephthalylidene dicamphor sulfonic acid, such as manufactured under the trademark “Mexoryl SX” by Chimex; and polyacrylamidomethyl benzylidene camphor, such as manufactured under the trademark “Mexoryl SW” by Chimex. Benzophenone compounds: Benzophenone-1 (2,4-dihydroxybenzophenone), such as marketed under the trademark “Uvinul 400” by BASF; benzophenone-2 (Tetrahydroxybenzophenone), such as marketed under the trademark “Uvinul D50” by BASF; Benzophenone-3 (2-hydroxy-4-methoxybenzophenone) or oxybenzone, such as marketed under the trademark “Uvinul M40” by BASF; benzophenone-4 (hydroxymethoxy benzophonene sulfonic acid), such as marketed under the trademark “Uvinul MS40” by BASF; benzophenone-5 (Sodium hydroxymethoxy benzophenone Sulfonate); benzophenone-6 (dihydroxy dimethoxy benzophenone); such as marketed under the trademark “Helisorb 11” by Norquay; benzophenone-8, such as marketed under the trademark “Spectra-Sorb UV-24” by American Cyanamid; benzophenone-9 (Disodium dihydroxy dimethoxy benzophenonedisulfonate), such as marketed under the trademark “Uvinul DS-49” by BASF; and benzophenone-12, and n-hexyl 2-(4-diethylamino-2-hydroxybenzoyl)benzoate (such as UVINUL A+by BASF). β,β-Diphenylacrylate compounds: Octocrylene, such as marketed in particular under the trademark “Uvinul N539” by BASF; and Etocrylene, such as marketed in particular under the trademark “Uvinul N35” by BASF. Triazine compounds: Diethylhexyl butamido triazone, such as marketed under the trademark “Uvasorb HEB” by Sigma 3V; 2,4,6-tris(dineopentyl 4′-aminobenzalmalonate)-s-triazine, bis-ethylhexyloxyphenol methoxyphenyl triazine, such as marketed under the trademark «TINOSORB S» by CIBA GEIGY, and ethylhexyl triazone, such as marketed under the trademark «UVTNUL T150» by BASF. Benzotriazole compounds, in particular, phenylbenzotriazole derivatives: 2-(2H-benzotriazole-2-yl)-6-dodecyl-4-methylpheno, branched and linear; and those described in U.S. Pat. No. 5,240,975. Benzalmalonate compounds: Dineopentyl 4′-methoxybenzalmalonate, and polyorganosiloxane comprising benzalmalonate functional groups, such as polysilicone-15, such as marketed under the trademark “Parsol SLX” by Hoffmann-LaRoche. Benzimidazole compounds, in particular, phenylbenzimidazole derivatives: Phenylbenzimidazole sulfonic acid, such as marketed in particular under the trademark “Eusolex 232” by Merck, and disodium phenyl dibenzimidazole tetrasulfonate, such as marketed under the trademark “Neo Heliopan AP” by Haarmann and Reimer. Imidazoline compounds: Ethylhexyl dimethoxybenzylidene dioxoimidazoline propionate. Bis-benzoazolyl compounds: The derivatives as described in EP-669,323 and U.S. Pat. No. 2,463,264. Para-aminobenzoic acid compounds: PABA (p-aminobenzoic acid), ethyl PABA, Ethyl dihydroxypropyl PABA, pentyl dimethyl PABA, ethylhexyl dimethyl PABA, such as marketed in particular under the trademark “Escalol 507” by ISP, glyceryl PABA, and PEG-25 PABA, such as marketed under the trademark “Uvinul P25” by BASF. Methylene bis-(hydroxyphenylbenzotriazol) compounds, such as 2,2′-methylenebis[6-(2H-benzotriazol-2-yl)-4-methyl-phenol], such as marketed in the solid form under the trademark “Mixxim BB/200” by Fairmount Chemical, 2,2′-methylenebis[6-(2H-benzotriazol-2-yl)-4-(1,1,3,3-tetramethylbutyl)phenol] such as marketed in the micronized form in aqueous dispersion under the trademark “Tinosorb M” by BASF, or under the trademark “Mixxim BB/100” by Fairmount Chemical, and the derivatives as described in U.S. Pat. Nos. 5,237,071 and 5,166,355, GB-2,303,549, DE-197,26,184, and EP-893,119, and Drometrizole trisiloxane, such as marketed under the trademark “Silatrizole” by Rhodia Chimie or—“Mexoryl XL” by L'Oreal. Benzoxazole compounds: 2,4-bis[5-I(dimethylpropyl)benzoxazol-2-yl-(4-phenyl)imino]-6-(2-ethylhexyl)imino-1,3,5-triazine, such as marketed under the trademark of Uvasorb K2A by Sigma 3V. Screening polymers and screening silicones: The silicones described in WO 93/04665. Dimers derived from a-alkylstyrene: The dimers described in DE-19855649. 4,4-Diarylbutadiene compounds: 1,1-dicarboxy(2,2′-dimethylpropyl)-4,4-diphenylbutadiene.

It is in some embodiments desirable that the organic UV filter(s) be selected from the group consisting of: butyl methoxydibenzoylmethane, ethylhexyl methoxycinnamate, homosalate, ethylhexyl salicylate, octocrylene, phenylbenzimidazole sulfonic acid, benzophenone-3, benzophenone-4, benzophenone-5, n-hexyl 2-(4-diethylamino-2-hydroxybenzoyl)benzoate, l,r-(1,4-piperazinediyl)bis[14244-(diethylamino)-2-hydroxybenzoyl]phenyl]-methanone 4-methylbenzylidene camphor, terephthalylidene dicamphor sulfonic acid, disodium phenyl dibenzimidazole tetrasulfonate, ethylhexyl triazone, bis-ethylhexyloxyphenol methoxyphenyl triazine, diethylhexyl butamido triazone, 2,4,6-tris(dineopentyl 4′-aminobenzalmalonate)-s-triazine, 2,4,6-tris(diisobutyl 4′-aminobenzalmalonate)-s-triazine, 2,4-bis-(n-butyl 4′-aminobenzalmalonate)-6-[(3-{1,3,3,3-tetramethyl-1-[(trimethylsilyloxy]-disiloxanyl}propyl)amino]-s-triazine, 2,4,6-tris-(di-phenyl)-triazine, 2,4,6-tris-(ter-phenyl)-triazine, methylene bis-benzotriazolyl tetramethylbutylphenol, drometrizole trisiloxane, polysilicone-15, dineopentyl 4′-methoxybenzalmalonate, 1,1-dicarboxy(2,2′-dimethylpropyl)-4,4-diphenylbutadiene, 2,4-bis[5-I (dimethylpropyl)benzoxazol-2-yl-(4-phenyl)imino]-6-(2-ethylhexyl)imino-1,3,5-triazine, camphor benzylkonium methosulfate, and mixtures thereof.

### Fatty Compounds

In addition to the constituents of the cosmetic composition described above, the composition of the present invention can also comprise additional fatty compounds selected from oils, waxes, fatty acids, fatty alcohols, and mixtures thereof. As used herein, the term “additional fatty compounds” does not include the carnauba wax described above.

The waxes useful for the present invention may be of mineral, fossil, animal, or vegetable origin, hydrogenated oils, or mixtures thereof. Non-limiting examples of waxes include hydrocarbon-based waxes such as beeswax, white beeswax, carnauba wax, candelilla wax, ouricury wax, Japan wax, cork fiber waxes or sugar cane waxes, paraffin waxes, lignite waxes, microcrystalline waxes, lanolin wax, montan wax, ozokerites, synthetic wax, polyethylene waxes, the waxes obtained by Fischer-Tropsch synthesis, hydrogenated oils and glycerides that are solid at 25° C. It is also possible to use silicone waxes, among which mention may be made of alkyl, alkoxy and/or esters of polymethylsiloxane.

Oils which can be used in the invention, mention may be made to polar or slightly polar oils, i.e. oils including an alkyl chain, preferably a C₃-C₄₀ alkyl chain. Non-limiting examples of oils to be used in the present invention include:

linear or branched hydrocarbons such as liquid paraffin, isohexadecane, liquid petroleum jelly and light naphthalene oils, and lanolin, hydrocarbon-based oils of plant origin, such as glyceride triesters, which are generally triesters of fatty acids and of glycerol, the fatty acids of which can have varied chain lengths from C₄ to C₂₄, it being possible for these chains to be saturated or unsaturated and linear or branched; these oils are in particular wheat germ oil, sunflower oil, grape seed oil, sesame oil, corn oil, apricot oil, castor oil, shea oil, avocado oil, olive oil, soybean oil, sweet almond oil, palm oil, rapeseed oil, cottonseed oil, hazelnut oil, macadamia oil, jojoba oil, alfalfa oil, poppy oil, pumpkin seed oil, marrow oil, blackcurrant oil, evening primrose oil, millet oil, barley oil, quinoa oil, rye oil, safflower oil, candlenut oil, passionflower oil and musk rose oil; or also caprylic/capric acid triglycerides,
synthetic esters, for instance oils of formula RCOOR′ in which R represents a linear or branched fatty acid residue containing from 1 to 40 carbon atoms and R′ represents a hydrocarbon-based chain that is especially branched, containing from 1 to 40 carbon atoms, on condition that R+R′ is ≥10, for instance, cetearyl octanoate, isopropyl myristate, isopropyl palmitate, C₁₂-C₁₅ alkyl benzoate, 2-ethylphenyl benzoate, isopropyl lanolate, hexyl laurate, diisopropyl adipate, isononyl isononanoate, oleyl erucate, 2-ethylhexyl palmitate, isostearyl isostearate, diisopropyl sebacate, octanoates, decanoates or ricinoleates of alcohols or polyalcohols, such as propylene glycol dioctanoate; hydroxylated esters, such as isostearyl lactate or diisostearyl malate; and pentaerythritol esters; citrates or tartrates, such as di(linear C₁₂-C₁₃ alkyl) tartrates, and also di(linear C₁₄-C₁₅ alkyl) tartrates, or acetates.
silicone oils such as polydimethylsiloxanes (PDMS's), optionally including a C₃-C₄₀ alkyl or alkoxy chain or a phenyl chain, such as phenyltrimethicones, optionally fluorinated polyalkylmethylsiloxanes, such as polymethyltrifluoropropyldimethylsiloxanes, or with functional groups such as hydroxyl, thiol and/or amine groups; polysiloxanes modified with fatty acids, fatty alcohols or polyoxyalkylenes, fluorosilicones and perfluoro oils;
mixtures thereof.

Non-liming examples of fatty alcohols useful for the present invention are those liquid at room temperature, containing a branched and/or unsaturated carbon-based chain containing from 12 to 26 carbon atoms, for instance octyldodecanol, isostearyl alcohol, oleyl alcohol, 2-hexyldecanol, 2-butyloctanol or 2-undecylpentadecanol.

Non-limiting examples of fatty acids useful for the present invention are higher fatty C₁₂-C₂₂ acids, such as oleic acid, linoleic acid or linolenic acid.

In a preferred embodiment, the fatty compounds of the cosmetic composition of the present invention are selected from the group consisting of waxes, linear or branched C₃-C₄₀ hydrocarbons, or mixtures thereof.

Preferably, the amount of fatty compounds in the cosmetic composition according to the present invention ranges from about 1% to about 30% by weight, more preferably from about 1% to about 20% by weight, more preferably from about 1% to about 15% by weight, most preferably from about 1% to about 10% by weight, based on the total weight of the composition.

### Additional Ingredients

In addition to the essential components described hereinbefore, the composition of the invention may further comprise any usual cosmetically acceptable ingredient, which may be chosen especially from perfume/fragrance, preserving agents, solvents, actives, vitamins, fillers, silicones, polymers, and mixtures thereof.

A person skilled in the art will take care to select the optional additional ingredients and/or the amount thereof such that the advantageous properties of the composition according to the invention are not, or are not substantially, adversely affected by the envisaged addition.

Suitable polymers include, but are not limited to xanthan gum, poly C₁₀₋₃₀ alkyl acrylate, acrylates/Ci 0-30 alkyl acrylate crosspolymer, styrene/acrylates copolymer, and mixtures thereof.

Non-limiting example of preserving agent which can be used in accordance with the invention include phenoxyethanol.

Suitable fillers of the invention could be as examples of oil-absorbing fillers: mica, silica, magnesium oxide, nylon-12, nylon-66, cellulose, talc, talc (and) methicone, talc (and) dimethicone, perlite, sodium silicate, pumice, PTFE, polymethyl methacrylate, alumina, calcium sodium borosilicate, magnesium carbonate, dimethicone/vinyl dimethicone crosspolymer.

Suitable solvents include, but are not limited to water, alcohols, glycols and polyols such as glycerin, caprylyl glycol, pentylene glycol, propylene glycol, butylene glycol, and mixtures thereof.

In various embodiments, the solvent is present in a concentration from about 15 to 90% by weight, or from about 20 to about 85% by weight, or from about 30 to about 75% by weight, or from about 35 to about 75% by weight, or preferably from about 40 to about 75% by weight, and more preferably from about 45 to about 75% by weight, including ranges and sub-ranges there between, based on the total weight of the combinations and/or compositions of the present disclosure.

Suitable additional actives include, but are not limited to, disodium EDTA, triethanolamine, and mixtures thereof.

Examples of silicones used in the composition of the present invention but not limited to are dimethicone and caprylyl methicone.

Non-limiting example of vitamins suitable for the composition of the present invention includes tocopherol.

Exemplary of polymers, include, but not limited to octenylsuccinate, xanthan gam, acrylates/C₁₀₋₃₀ alkyl acrylate crosspolymer and styrene/acrylates copolymer.

The additional ingredients, including the solvents, may represent from 20% to 90%, such as from 25% to 85% or such as from 30 to 80% by weight, based on the total weight of the composition of the invention.

By way of non-limiting illustration, the invention will now be described with reference to the following examples.

### EXAMPLES

### Examples 1 to 3

Suitable compositions according to the present invention are as Examples 1 to 3, as follows:

| FUNCTION | INGREDIENT | EX. 1 | EX. 2 | EX. 3 |
|---|---|---|---|---|
| NONIONIC | STEARETH-2 | 2.0 | 4.0 | 2.5 |
| SURFACTANT | PEG-100 STEARATE | 1.0 | 1.5 | 0.5 |
| | STEARETH-20 | 0.8 | 0.5 | 0.5 |
| | CETEARYL ALCOHOL | 3.0 | 1.0 | 1.0 |
| | GLYCERYL STEARATE | 1.0 | 3.0 | 0.5 |
| HYDROPHOBIC | POLYLACTIC ACID | 0.0 | 4.0 | 0.0 |
| COMPOUND | SILICA SILYLATE | 0.0 | 0.0 | 1.5 |
| WATER-SOLUBLE | HYDROXYPROPYL STARCH | 1.5 | 2.0 | 3.0 |
| POLYSACCHARIDE | PHOSPHATE | | | |
| UV FILTER | BUTYL | 2.0 | 5.0 | 5.5 |
| | METHOXYDIBENZOYLMETHANE | | | |
| | ETHYLHEXYL SALICYLATE | 1.0 | 1.0 | 2.0 |
| | ETHYLHEXYL TRIAZONE | 4.0 | 5.0 | 3.0 |
| | OCTOCRYLENE | 6.0 | 7.0 | 2.0 |
| | HOMOSALATE | 2.0 | 4.0 | 6.0 |
| FATTY | BEESWAX | 2.0 | 3.0 | 1.5 |
| COMPOUND | CAPRYLIC/CAPRIC | 3.0 | 1.0 | 7.0 |
| | TRIGLYCERIDE | | | |
| ADDITIONAL | PHENOXYETHANOL | 1.0 | 1.5 | 0.5 |
| COMPOUND | DIMETHICONE | 12.0 | 6.0 | 8.0 |
| SOLVENT | CAPRYLYL GLYCOL | 0.5 | 0.2 | 0.8 |
| | WATER | Q.S. | Q.S. | Q.S. |

### Examples 4 and 5

Compositions according to the prior art are as Examples 4 and 5, as follows:

| FUNCTION | INGREDIENT | EX. 4 | EX. 5 |
|---|---|---|---|
| SURFACTANT | STEARIC ACID | 3.0 | 1.8 |
| | GLYCERYL STEARATE (and) PEG-100 | 1.0 | 0.4 |
| | STEARATE | | |
| | GLYCERYL ISOSTEARATE | 0.0 | 2.8 |
| | POTASSIUM CETYL PHOSPHATE | 2.0 | 0.7 |
| | POLOXAMER 338 | 0.5 | 0.0 |
| | STEARYL ALCOHOL | 0.2 | 1.7 |
| | SODIUM METHYL STEAROYL TAURATE | 0.7 | 0.0 |
| HYDROPHOBIC | POLYLACTIC ACID | 0.2 | 0.0 |
| COMPOUND | SILICA | 4.4 | 0.0 |
| | SILICA SILYLATE | 0.1 | 0.0 |
| UV FILTER | PHENYLBENZIMIDAZOLE SULFONIC | 2.9 | 0.0 |
| | ACID | | |
| | BUTYL METHOXYDIBENZOYLMETHANE | 2.4 | 4.0 |
| | ETHYLHEXYL SALICYLATE | 0.0 | 4.0 |
| | TITANIUM DIOXIDE | 1.0 | 2.0 |
| | ETHYLHEXYL TRIAZONE | 5.0 | 0.9 |
| | TEREPHTHALYLIDENE DICAMPHOR | 2.5 | 3.1 |
| | SULFONIC ACID | | |
| | OCTOCRYLENE | 2.0 | 4.0 |
| | HOMOSALATE | 2.3 | 5.0 |
| | BIS-ETHYLHEXYLOXYPHENOL | 3.6 | 0.0 |
| | METHOXYPHENYL TRIAZINE | | |
| | SILICA (and) TITANIUM DIOXIDE | 4.5 | 0.0 |
| | METHYLENE BIS-BENZOTRIAZOLYL | 3.2 | 0.0 |
| | TETRAMETHYLBUTYLPHENOL (and) | | |
| | POLYGLYCERYL-10 LAURATE | | |
| FATTY | ISONONYL ISONONANOATE | 4.5 | 0.0 |
| COMPOUND | DIISOPROPYL SEBACATE | 0.5 | 0.0 |
| ADDITIONAL | DISODIUM EDTA | 0.1 | 0.2 |
| COMPOUND | TRIETHANOLAMINE | 2.5 | 0.4 |
| | TALC | 0.0 | 2.0 |
| | PHENOXYETHANOL | 1.8 | 1.5 |
| | FRAGRANCE | 0.0 | 0.2 |
| | ALUMINUM STARCH | 2.7 | 0.0 |
| | OCTENYLSUCCINATE | | |
| | ACRYLATES/C10-30 ALKYL ACRYLATE | 0.0 | 0.2 |
| | CROSSPOLYMER | | |
| | ACRYLATES/C10-30 ALKYL ACRYLATE | 0.1 | 0.0 |
| | CROSSPOLYMER | | |
| | SYNTHETIC WAX | 0.0 | 1.0 |
| | AMMONIUM | 1.8 | 0.0 |
| | ACRYLOYLDIMETHYLTAURATE/VP | | |
| | COPOLYMER | | |
| | CYCLOHEXASILOXANE | 0.0 | 1.0 |
| | CAPRYLYL METHICONE | 3.0 | 0.0 |
| | TOCOPHEROL | 1.0 | 0.1 |
| SOLVENT | ALCOHOL | 0.0 | 4.0 |
| | WATER | Q.S. | Q.S. |
| | GLYCERIN | 1.5 | 1.0 |
| | PENTYLENE GLYCOL | 3.0 | 0.0 |
| | CAPRYLYL GLYCOL | 1.0 | 2.0 |

### Example 6

The composition according to the present invention and the compositions according to the prior art were submitted to rheology tests so as to compare their rheological behavior. Particularly, a viscosity profiling and a strain sweep test were performed.

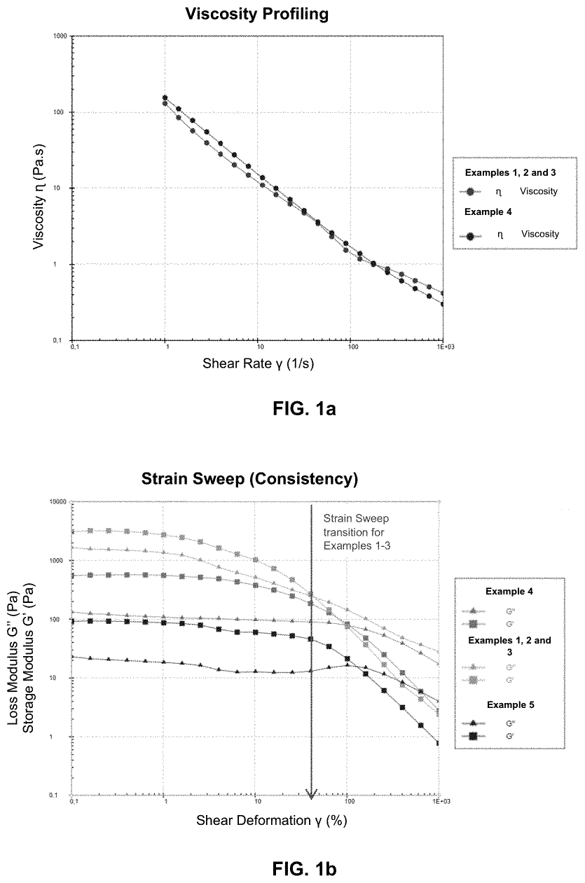

From the results shown in FIG. 1, it is possible to conclude that both compositions according to examples 1 to 3 have the same viscosity behavior as the composition of example 4 (FIG. 1a), non-Newtonian fluids. Regarding the consistency, shown in FIG. 1b, the storage modulus G′ and the loss modulus G″ values, it is possible to conclude that the composition according to the present invention (example 1) presents a higher consistency than the compositions according to the state of the art (examples 4 and 5).

It is demonstrated that with the same viscosity behavior it is possible to achieve different yield stress point and strain sweep transition, which brings an interesting texture property, as better spreadability, to the composition according to the present invention.

1. A cosmetic composition comprising:
(a) at least 1% by weight, based on the total weight of the composition, of at least one nonionic surfactant;
(b) at least 0.5% by weight, based on the total weight of the composition of a hydrophobic powder selected from the group consisting of silica aerogel and polylactic acid (PLA);
(c) at least 1% by weight, based on the total weight of the composition, of at least one water-soluble polysaccharide chosen from starches; and
(d) UV filter system,
wherein the composition is in a soft-solid format.

2. The cosmetic composition, according to claim 1, comprising from 1% to 20% by weight of the at least one nonionic surfactant, based on the total weight of the composition.

3. The cosmetic composition, according to claim 1, comprising from 1% to 10% by weight of the hydrophobic powder, based on the total weight of the composition.

4. The cosmetic composition, according to claim 1, comprising from 0.5% to 10% by weight of the at least one water-soluble polysaccharide, based on the total weight of the composition.

5. The cosmetic composition, according to claim 1, wherein the UV filter system in the soft-solid cosmetic composition ranges from 0.1% to 50% by weight, based on the total weight of the composition.

6. The cosmetic composition, according to claim 1, wherein the at least one non-ionic surfactant is selected from the group consisting of steareth-2, steareth-20, glyceryl stearate, PEG-100 stearate and cetearyl alcohol.

7. The cosmetic composition, according to claim 1, wherein the at least one water-soluble polysaccharide is selected from the group consisting of monostarch phosphates, distarch phosphates and tristarch phosphates.

8. The cosmetic composition, according to claim 7, wherein the at least one water-soluble polysaccharide is hydroxypropyl starch phosphate.

9. The cosmetic composition, according to claim 1, wherein the UV filter system comprises at least one UV filter selected from the group of inorganic UV filters and organic UV filters.

10. The cosmetic composition, according to claim 8, wherein the UV filter system comprises at least one organic UV filter selected from the group consisting of anthranilic compounds; dibenzoylmethane compounds; cinnamic compounds; salicylic compounds; camphor compounds; benzophenone compounds; β,β-diphenylacrylate compounds; triazine compounds; benzotriazole compounds; benzalmalonate compounds; benzimidazole compounds; imidazoline compounds; bis-benzoazolyl compounds; p-aminobenzoic acid (PABA) compounds; methylenebis(hydroxyphenylbenzotriazole) compounds; benzoxazole compounds; screening polymers and screening silicones; dimers derived from a-alkylstyrene; 4,4-diarylbutadienes compounds; guaiazulene and derivatives thereof; rutin and derivatives thereof; flavonoids; bioflavonoids; oryzanol and derivatives thereof; quinic acid and derivatives thereof; phenols; retinol; cysteine; aromatic amino acids; peptides having an aromatic amino acid residue; and mixtures thereof.

11. The cosmetic composition, according to claim 10, wherein the UV filter system comprises at least one organic UV filter selected from the group consisting of butyl methoxydibenzoylmethane, ethylhexyl salicylate, ethylhexyl triazone, octocrylene and homosalate.

12. The cosmetic composition, according to claim 11, wherein the UV filter system comprises at least one inorganic UV filter selected from the group consisting of silicon carbide, coated or uncoated metal oxides, and mixtures thereof.

13. The cosmetic composition, according to claim 1, further comprising fatty compounds selected from the group consisting of waxes, linear or branched C₃-C₄₀ hydrocarbons, or mixtures thereof.

14. The cosmetic composition, according to claim 13, wherein the fatty compounds in the soft-solid cosmetic composition ranges from 1% to 30% by weight, based on the total weight of the composition.

15. The cosmetic composition, according to claim 1, further comprising at least one anionic surfactant.

16. Use of a cosmetic composition comprising:
(a) at least 1% by weight, based on the total weight of the composition, of at least one nonionic surfactant;
(b) at least 0.5% by weight, based on the total weight of the composition of a hydrophobic powder selected from the group consisting of silica aerogel and polylactic acid (PLA);
(c) at least 1% by weight, based on the total weight of the composition, of at least one water-soluble polysaccharide chosen from starches; and
(d) UV filter system,
for the manufacture of a product to be used as sunscreen daily product, wherein the composition is in a soft-solid format.
